SEQUENCE LISTING (21 sequences)

SEQ ID NO: 1 - protein, 97 aa, synthetic construct
ALSYDTEILTVEYGFLPIGXIVEEXIEXTVYSVDXXGFVYTQPIAQWHNRGEQEVFEYXLEDGSIIRATXDHXFMTTDGXMLPIDEIFEXGLDLXQV

SEQ ID NO: 2 - protein, 97 aa, synthetic construct
ALSYDTEILTVEYGFLPIGRIVEENIEATVYSVDRHGFVYTQPIAQWHNRGEQEVFEYDLEDGSIIRATRDHRFMTTDGEMLPIDEIFEQGLDLRQV

SEQ ID NO: 3 - protein, 20 aa, synthetic construct
DADSGGSGKDDAKKKDDTKK

SEQ ID NO: 4 - protein, 16 aa, synthetic construct
GGSGKDDAKKKDDTKK

SEQ ID NO: 5 - protein, 16 aa, synthetic construct
DADSKDDAKKKDDTKK

SEQ ID NO: 6 - protein, 12 aa, synthetic construct
KDDAKKKDDTKK

SEQ ID NO: 7 - protein, 14 aa, synthetic construct
ARTKQTARKSTGGK

SEQ ID NO: 8 - protein, 18 aa, synthetic construct
ARTKQTARKSTGGKAPRK

SEQ ID NO: 9 - protein, 16 aa, synthetic construct
SGRGKGGKGLGKGGAK

SEQ ID NO: 10 - protein, 13 aa, synthetic construct
AKSAPAPKKGSKK

SEQ ID NO: 11 - protein, 12 aa, synthetic construct
PKKTESSKSKSK

SEQ ID NO: 12 - protein, 14 aa, synthetic construct
ARTKQTARKSTGGR

SEQ ID NO: 13 - protein, 9 aa, synthetic construct
DADSGGSGK

SEQ ID NO: 14 - protein, 15 aa, synthetic construct
DADSGGSGKDDAKKK

SEQ ID NO: 15 - protein, 10 aa, synthetic construct
DADSGGSGDK

SEQ ID NO: 16 - protein, 14 aa, synthetic construct
DADSGGSGKDDTKK

SEQ ID NO: 17 - protein, 14 aa, synthetic construct
ARTRQTARKSTGGK

SEQ ID NO: 18 - protein, 14 aa, synthetic construct
ARTKQTARRSTGGK

SEQ ID NO: 19 - protein, 8 aa, synthetic construct
DYKDDDDK

SEQ ID NO: 20 - protein, 117 aa, synthetic construct
ALSYDTEILTVEYGFLPIGRIVEENIEATVYSVDRHGFVYTQPIAQWHNRGEQEVFEYDLEDGSIIRATRDHRFMTTDGEMLPIDEIFEQGLDLRQVDADSGGSGKDDAKKKDDTKK

SEQ ID NO: 21 - protein, 97 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRV

(19) United States
(12) Patent Application Publication
(10) Pub. No.: US 2026/0297126 A1
(43) Pub. Date: Oct. 1, 2026

(54) SPLIT INTEINS FOR AFFINITY CAPTURE

(71) Applicant: Cytiva BioProcess R&D AB, Uppsala (SE)

(72) Inventors: Johan F. Öhman, Uppsala (SE); Peter Lundbäck, Uppsala (SE)

(21) Appl. No.: 19/480,329

(22) PCT Filed: Apr. 24, 2024
(86) PCT No.: PCT/EP2024/061173

§ 371 Date: Oct. 30, 2025

(30) Foreign Application Priority Data
May 8, 2023 (SE) 2350555-5

(51) Int. Cl. C07K 1/22

(52) U.S. Cl. CPC C07K 1/22

(57) ABSTRACT

Disclosed herein is a protein for affinity capture, the protein comprising: a ligand moiety capable of affinity interaction with a target molecule, and a linker moiety bound to the ligand moiety and capable of being coupled to a solid phase, wherein the ligand moiety is free from lysine residues, and wherein the linker moiety is terminally positioned in the protein and has at least two lysine residues, wherein at least one residue other than lysine is terminally arranged in relation to at least two of the lysine residues. Further disclosed is an affinity capture medium comprising the protein, a split intein system for affinity capture of a protein of interest, and a method for purification of a protein of interest.

### FIELD OF INVENTION

The present invention relates to a protein for affinity capture. The present invention further relates to an affinity capture medium comprising the protein, a split intein system for affinity capture comprising the protein, and a method for purification of a protein of interest.

### BACKGROUND

Protein immobilization on solid phases is important in many biotechnological applications, for example in development of biosensors, immunoassays and bioseparations where affinity based separation is taken advantage of. In affinity separation, it is desirable, but problematic, to attach ligands in form of proteins a solid phase without causing denaturation of the protein, or steric hindrance and blocking of active sites of the protein.

It has been suggested that immobilization of proteins to a solid phase with a preferable orientation can avoid denaturation of the protein, prevent steric hindrance and keep active sites exposed to the solution and thus preserve the bioaffinity or bioactivity (Wei Huang et al., Anal. Chem., 69 (22), 4601-4607, 1997).

It has further been suggested that problems associated with denaturation of the protein, steric hindrance or blocking of active sites can be reduced by inserting a linker between the protein and the site used for attachment to the solid phase. Peptide linkers occur naturally in multidomain proteins, where they serve as spacers, but they can also be used for the immobilization of proteins to a solid phase. It is however problematic to achieve a high degree of immobilisation of the proteins to the solid phase.

It is further problematic to achieve immobilisation of the protein specifically via the linker, i.e. to avoid that the protein is directly linked to the solid phase. Yet further, it is problematic to achieve a high immobilisation density on the surface of the solid phase.

It would be highly desirable to provide ligands for affinity separation, or affinity capture, in form of proteins, such as inteins, that could be bound to a solid phase in a predictable manner, at high surface density, and that would provide for efficient and/or improved affinity separation or capture.

### SUMMARY OF INVENTION

An object of the present invention is to, at least partially, overcome problems or disadvantages of prior art.

According to a first aspect, there is provided a protein for affinity capture, the protein comprising: a ligand moiety capable of affinity interaction with a target molecule, and a linker moiety bound to the ligand moiety and capable of being coupled to a solid phase, wherein the ligand moiety is free from lysine residues, and wherein the linker moiety is terminally positioned in the protein and has at least two lysine residues, wherein, in the linker moiety, at least one residue other than lysine is terminally arranged in relation to at least two of the lysine residues.

The ligand moiety being capable of affinity interaction with a target molecule allows for affinity based binding, purification or separation of the target molecule, such as a protein of interest.

The protein having a linker moiety bound to the ligand moiety and capable of being coupled to a solid phase allows for immobilisation of the protein to a solid phase, and, thus, allows for provision of solid phase materials or medium for affinity chromatography. Further, the linker moiety allows for a physical distance between the ligand moiety and the solid phase, thus reducing risks of deformation, such as denaturation, of the ligand moiety, when immobilised on a solid phase, thereby allowing for improved affinity interaction and separation. Yet further, such a physical distance between the ligand moiety and the solid phase improves access of active sites of the ligand moiety, such as affinity sites, e.g. from reduced steric hindrance by the solid phase, thereby allowing for improved affinity interaction separation.

The ligand moiety being free from lysine residues provides for improved binding to the solid phase via the linker moiety. Specific binding between the linker moiety and the solid phase may be realised via covalent binding between the solid phase and lysine while direct binding between the ligand moiety and the solid phase may be avoided.

The linker moiety having at least two lysine residues, allows for linking or binding of the linker moiety, and thereby the ligand moiety, to the solid phase via at least one lysin residue. Thereby, improved control of binding to the solid phase may be realised.

Having at least two lysine residues, wherein, in the linker moiety, at least one residue other than lysine is terminally arranged in relation to at least two of the lysine residues, provides for improved binding to the solid phase, as will be evident from experimental data provided herein.

The linker moiety may comprise or consist of an amino acid sequence having 5 to 100 amino acid residues.

The linker moiety may have an isoelectric point above 6.0. Thereby, improved immobilisation to the solid phase may be realised.

The linker moiety may have an isoelectric point above 8.0.

The linker moiety may have at least three lysine residues. Thereby, improved binding to the solid phase may be realised.

If the linker moiety has three lysine residues, the linker moiety may have an isoelectric point above 8.

The ligand moiety may be a split N-intein or a split C-intein, thus capable of affinity interaction with a corresponding split C-intein or split N-intein, respectively.

The ligand moiety may be a split N-intein and the linker moiety may be a C-terminally arranged linker moiety, wherein the split N-intein comprises the following sequence:

(SEQ ID NO: 1)
ALSYDTEILTVEYGFLPIGXIVEEXIEXTVYSVDXXGFVYTQPIAQWHN
RGEQEVFEYXLEDGSIIRATXDHXFMTTDGXMLPIDEIFEXGLDLXQV,

wherein,
X in positions 20, 35, 70, 73, and 95 are each independently selected from R or A;
X in position 28 is C, A or S;
X in position 36 is N, H or Q;
X in position 25 is N or R;
X is position 59 is D or C;
X in position 80 is E or Q; and
X in position 90 is Q, or R.

It is beneficial that such a split N-intein is free from lysine residues. When combined with the linker moiety having at least two lysine residues, specific binding to the solid phase via the linker moiety may be realised, in particular when at least one residue other than lysine is terminally arranged in relation to at least two of the lysine residues. Thereby, the ligand may be immobilised to the solid phase while reducing risks of deformation or reduced affinity activity of the ligand moiety, allowing for improved affinity interaction and separation.

Of SEQ ID NO: 1,
X in positions 20, 35, 70, 73, and 95 may be R;
X in position 28 may be A;
X in position 36 may be H;
X in position 25 may be N;
X in position 59 may be D;
X in position 80 may be E; and
X in position 90 may be Q.
   Thus, the split N-intein may comprises or consist of the sequence defined by SEQ ID NO: 2.

The linker moiety may comprise an amino acid sequence selected from the list consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12. All of those sequences (SEQ ID NO: 3-12) comprises at least two lysine residues, wherein at least one residue other than lysine is terminally arranged in relation to at least two of the lysine residues, which provides for improved immobilisation to the solid phase.

The linker moiety may comprise an amino acid sequence having at least 80%, such as at least 90%, or 90-100%, identity with any one of SEQ ID NO: 3-12, provided that the linker moiety has at least two lysine residues, wherein at least one residue other than lysine is terminally arranged in relation to at least two of the lysine residues.

According to a second aspect, there is provided an affinity capture medium comprising the protein for affinity capture according to the first aspect and a solid phase, wherein the ligand moiety of the protein is covalently attached to the solid phase via the linker moiety.

The linker moiety of the protein may be covalently attached to the solid phase via at least one lysine residue of the amino acid sequence of the linker moiety.

The solid phase may be in form of a bead, a membrane, a particle, a chip, a fibrous or nanofibrous structure, or a monolithic structure, or combinations thereof.

The solid phase may comprise or be composed of a natural or synthetic polymeric material, preferably a polysaccharide such as cellulose or agarose.

According to a third aspect, there is provided a split intein system for affinity capture of a protein of interest (POI), comprising: a protein for affinity capture according to the first aspect, wherein the ligand is a split N-intein; and a target molecule which is a POI fused with a split C-intein.

The protein for affinity capture may be comprised in an affinity capture medium according to the second aspect.

According to a fourth aspect, there is provided a method for purification of a protein of interest (POI). The method comprises: loading a sample liquid comprising the POI on an affinity capture medium comprising a protein for affinity capture, having affinity for the POI or for a target molecule comprising the POI, covalently attached to a solid phase via a linker moiety, wherein the protein for affinity capture is the protein according to the first aspect; capturing the POI or the target molecule comprising the POI by binding to the protein of the affinity capture medium; washing the affinity capture medium with washing liquid to remove compounds other than the POI or the target molecule comprising the POI; and eluting the POI or the target molecule comprising the POI from the affinity capture medium.

The affinity capture medium may be a separation medium.

The POI or the target molecule comprising the POI being eluted from the affinity capture medium is released from the protein of the affinity capture medium. Releasing the POI or the target molecule comprising the POI may occur in conjunction with the elution, such as initiated by elution liquid, and/or may occur separate from the elution.

The method may further comprise releasing the POI or the target molecule comprising the POI from the protein of the affinity capture medium; which releasing may occur prior to or simultaneous with the eluting the POI or the target molecule comprising the POI from the affinity capture medium, or a combination thereof. Releasing and/or elution of the POI or the target molecule comprising the POI from the protein may occur via different mechanisms, for example depending on the type of ligand, POI and/or target molecule. For example, the releasing or the elution may occur spontaneously, by use of additives or reagents, or by changes in properties of present buffer or liquid. Such properties may, for example, be pH or ionic or salt concentration, or hydrophobicity or hydrophilicity, or combinations of different such properties. Spontaneous release may be achieved, for example, using split inteins allowing for spontaneous cleavage, or self-cleavage, whereby the POI is cleaved off a split intein with which it is fused.

The affinity capture medium may be the affinity capture medium according to the second aspect.

Features of one aspect may be applicable to any one of the other aspects as well. In order to avoid undue repetition, reference is made between the aspects with regard to features, when applicable.

A further scope of applicability of the present disclosure will become apparent from the detailed description given below. However, it should be understood that the detailed description and specific examples, while indicating preferred variants of the present inventive concept, are given by way of illustration only, since various changes and modifications within the scope of the inventive concept will become apparent to those skilled in the art from this detailed description. Hence, it is to be understood that this inventive concept is not limited to the particular steps of the methods described or component parts of the systems described as such method and system may vary. It is also to be understood that the terminology used herein is for purpose of describing particular embodiments only and is not intended to be limiting. It must be noted that, as used in the specification and the appended claim, the articles “a”, “an”, “the”, and “said” are intended to mean that there are one or more of the elements unless the context clearly dictates otherwise. Furthermore, the words “comprising”, “including”, “containing” and similar wordings do not exclude other elements or steps.

### BRIEF DESCRIPTION OF THE DRAWINGS

Aspects of the present disclosure will now be described in more detail, with reference to the appended drawings showing variants of the invention. The figures should not be considered limiting the inventive concept to the specific variant; instead, they are used for explaining and understanding the inventive concept. As illustrated in the figures, the sizes of eg. moieties or solid phases may be schematical and may, for example, be exaggerated, for illustrative purposes and, thus, are provided to illustrate the general structures of variants of the present inventive concept. Like reference numerals refer to like elements throughout.

FIG. 1 illustrates a protein for affinity capture.

FIG. 2 illustrates a variant of a protein for affinity capture bound to a solid phase.

FIG. 3 illustrates an affinity capture medium.

FIGS. 4A and 4B each illustrates a split intein system.

FIG. 5 illustrates a method for purification of a protein of interest.

FIG. 6 illustrates a variant of a method for purification of a protein of interest.

FIG. 7 illustrates a diagram illustrating immobilisation levels.

FIG. 8 illustrates a diagram illustrating binding levels.

FIG. 9 illustrates a UV-chromatogram obtained from an experiments.

FIG. 10 illustrates a photograph of a SDS-PAGE gel from experiments.

### DETAILED DESCRIPTION

The present invention will now be described more fully hereinafter. This invention may, however, be embodied in many different forms and should not be construed as limited to the embodiments set forth herein; rather, these embodiments are provided for thoroughness and completeness, and for fully conveying the scope of the invention to a skilled person. Although individual features may be included in different embodiments, these may possibly be combined in other ways, and the inclusion in different embodiments does not imply that a combination of features is not feasible.

The present inventive concepts are, at least in part, based on a realisation that proteins for affinity capture, eg. proteins capable of affinity interactions with a target molecule, may be provided comprising a ligand moiety and a linker moiety, wherein the ligand moiety is free from lysine residues, while the linker moiety comprises at least two lysine residues, wherein, in the linker moiety, at least one residue other than lysine is terminally arranged in relation to at least two of the lysine residues, improved immobilisation of the protein to a solid phase is achievable.

Affinity capture shall be understood as comprising and referring to any suitable form of capturing based on affinity recognition or interaction, wherein the target molecule may be captured by or interact with the ligand moiety based on affinity recognition. Affinity capturing, as used herein, thus, may refer to or include affinity separation, such as affinity chromatography or affinity based purification, but may also refer to or include, for example, affinity sensoring, or affinity based assays. The protein for affinity capture, thus and for example, may be a protein for affinity separation.

The term “peptide”, “polypeptides” and “protein” are used herein and include proteins and fragments thereof. Peptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Peptides include any oligopeptide, polypeptide, gene product, expression product, or protein. A peptide is comprised of consecutive amino acids and encompasses naturally occurring or synthetic molecules. In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, linkage of distinct domains or motifs, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, acemization, selenoylation, sulfation, and transfer-RN A mediated addition of amino acids to protein such as arginylation. (See Proteins-Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “protein of interest (POI)” includes any synthetic or naturally occurring protein or peptide. The term therefore encompasses those compounds traditionally regarded as drugs, vaccines, and biopharmaceuticals including molecules such as proteins, peptides, and the like. Examples of therapeutic agents are described in well-known literature references such as the Merck Index (14th edition), the Physicians' Desk Reference (64^{th} edition), and The Pharmacological Basis of Therapeutics (1st edition), and they include, without limitation, medicaments; substances used for the treatment, prevention, diagnosis, cure or mitigation of a disease or illness; substances that affect the structure or function of the body, or pro-drugs, which become biologically active or more active after they have been placed in a physiological environment.

“Intein” refers to an in-frame intervening sequence in a protein. An intein can catalyze its own excision from the protein through a post-translational protein splicing process to yield the free intein and a mature protein. An intein can also catalyze the cleavage of the inteinextein bond at either the intein N-terminus, or the intein C-terminus, or both of the inteinextein termini. As used herein, “intein” encompasses mini-inteins, modified or mutated inteins, and split inteins. As used herein, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, may be used to derive a split intein for use in the proteins, systems and methods disclosed herein. For example, in one variant the split intein may be derived from a eukaryotic intein. According to another variant, the split intein may be derived from a bacterial intein. According to another variant, the split intein may be derived from an archaeal intein. The split intein so-derived may possess only the amino acid sequences essential for catalyzing splicing reactions. As used herein, the “split N-intein”, “N-terminal intein segment” or “N-intein” refers to any intein sequence that comprises an N-terminal amino acid sequence that is functional for splicing and/or cleaving reactions when combined with a corresponding C-terminal intein segment. An N-terminal intein segment. or split N-intein, thus also comprises a sequence that is spliced out when splicing occurs. A split N-intein may comprise a sequence that is a modification of the N-terminal portion of a naturally occurring (native) intein sequence. Non-intein residues can also be genetically fused to intein segments to provide additional functionality, such as the ability to be affinity purified or to be covalently immobilized. As used herein, the “split C-intein”, “C-terminal intein segment” or “C-intein” refers to any intein sequence that comprises a C-terminal amino acid sequence that is functional for splicing or cleaving reactions when combined with a corresponding N-terminal intein segment. According to a variant, the C-terminal intein segment may comprise a sequence that is spliced out when splicing occurs. In another variant, the split C-intein may be cleaved from a peptide sequence fused to its C-terminus. The sequence which is cleaved from the C-terminal intein's C-terminus may be referred to herein as a “protein of interest POI”. A C-terminal intein segment can comprise a sequence that is a modification of the C-terminal portion of a naturally occurring (native) intein sequence. For example, a C-terminal intein segment can comprise additional amino acid residues and/or mutated residues so long as the inclusion of such additional and/or mutated residues does not render the C-terminal intein segment non-functional for splicing or cleaving.

### N-Intein Protein Variants

Variants of aspects relates to affinity separation, such as affinity chromatography. Affinity tag cleavage mechanisms in a single step using a split intein system may be used according to variants of aspects herein that cleaves with broad amino acid tolerance to generate a tag less protein of interest (POI) as end product. The two halves of the intein may be the ligand moiety (such as a split N-intein) and an affinity tag (such as a split C-intein) fused with the target molecule, and they associate rapidly. Immobilizing one half (split N-intein) on a solid phase, such as a chromatography resin, enables the capture of the other half (split C-intein) fused or coupled to the POI from sample liquid, or a sample solution. With some inteins a presence of e.g. Zn²⁺ ions, may inhibit the cleavage reaction, enabling a stable complex to form, for example while impurities are washed away. With other inteins cleavage may be inhibited or promoted by changing other properties, such as, for example, pH or ionic strength. After impurities are eliminated or removed, a chelator or reducing agent may be added, and the cleavage reaction proceed, enabling collection of the POI, while the intein tag remains bound non-covalently to the cognate intein linked to the solid phase.

Native inteins are known in the art. A list of inteins is found in Table 1 of WO 2021/099607, herein included as reference. All inteins have the potential to be made into split inteins while some inteins naturally exist in split form.

The split inteins of variants of aspects herein, may be modified, or mutated, inteins. A modified intein may comprise modifications to the N-terminal intein segment, the C-terminal intein segment, or both. The modifications may include additional amino acids at the N-terminus the C-terminus of either portion of the split intein, or may be within the either portion of the split intein.

### Disclosure of Sequence Used Herein

Below list of sequences are disclosed and used herein.

| SEQ ID | SEQUENCE |
|---|---|
| SEQ ID NO: 1 | ALSYDTEILTVEYGFLPIGXIVEEXIEXTVYSV |
| | DXXGFVYTQPIAQWHNRGEQEVFEYXLEDGSII |
| | RATXDHXFMTTDGXMLPIDEIFEXGLDLXQV |
| SEQ ID NO: 2 | ALSYDTEILTVEYGFLPIGRIVEENIEATVYSV |
| | DRHGFVYTQPIAQWHNRGEQEVFEYDLEDGSII |
| | RATRDHRFMTTDGEMLPIDEIFEQGLDLRQV |
| SEQ ID NO: 3 | DADSGGSGKDDAKKKDDTKK |
| SEQ ID NO: 4 | GGSGKDDAKKKDDTKK |
| SEQ ID NO: 5 | DADSKDDAKKKDDTKK |
| SEQ ID NO: 6 | KDDAKKKDDTKK |
| SEQ ID NO: 7 | ARTKQTARKSTGGK |
| SEQ ID NO: 8 | ARTKQTARKSTGGKAPRK |
| SEQ ID NO: 9 | SGRGKGGKGLGKGGAK |
| SEQ ID NO: 10 | AKSAPAPKKGSKK |
| SEQ ID NO: 11 | PKKTESSKSKSK |
| SEQ ID NO: 12 | ARTKQTARKSTGGR |
| SEQ ID NO: 13 | DADSGGSGK |
| SEQ ID NO: 14 | DADSGGSGKDDAKKK |
| SEQ ID NO: 15 | DADSGGSGDK |
| SEQ ID NO: 16 | DADSGGSGKDDTKK |
| SEQ ID NO: 17 | ARTRQTARKSTGGK |
| SEQ ID NO: 18 | ARTKQTARRSTGGK |
| SEQ ID NO: 19 | DYKDDDDK |
| SEQ ID NO: 20 | ALSYDTEILTVEYGFLPIGRIVEENIEATVYSV |
| | DRHGFVYTQPIAQWHNRGEQEVFEYDLEDGSII |
| | RATRDHRFMTTDGEMLPIDEIFEQGLDLRQVDA |
| | DSGGSGKDDAKKKDDTKK |
| SEQ ID NO: 21 | CLSYETEILTVEYGLLPIGKIVEKRIECTVYSV |
| | DNNGNIYTQPVAQWHDRGEQEVFEYCLEDGSLI |
| | RATKDHKFMTVDGQMLPIDEIFERELDLMRV |

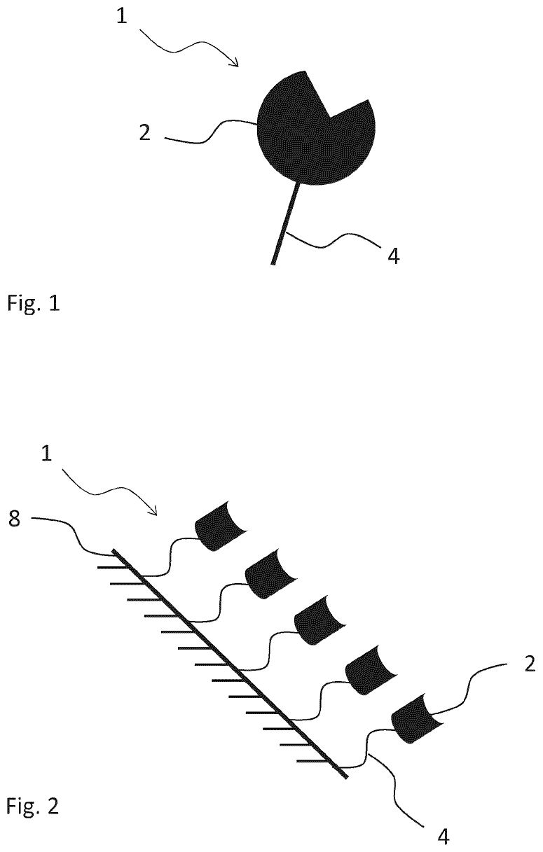

With reference to FIG. 1, a protein 1 for affinity capture will now be discussed. The protein 1 may be a protein 1 in accordance with the first aspect or variants thereof. FIG. 1 is a schematic illustration presented in an attempt to improve understanding of the protein 1 as it may be used according to aspects herein. The protein 1 comprises a ligand moiety 2 capable of affinity interaction with a target molecule, and a linker moiety 4 bound to the ligand moiety 2 and capable of being coupled to a solid phase (not illustrated). Further, the ligand moiety 2 is free from lysine residues, and the linker moiety 4 is terminally positioned in the protein 1 and has at least two lysine residues, wherein, in the linker moiety, at least one residue other than lysine is terminally arranged in relation to at least two of the lysine residues. Concerning the at least one residue other than lysine being terminally arranged in relation to at least two of the lysine residues, terminally arranged refers to and is to be understood as being arranged at the terminal end being distal in relation to the ligand moiety.

The ligand moiety may comprise a polypeptide. In particular, the ligand moiety may consist of a polypeptide.

The linker moiety may comprise a polypeptide. In particular, the linker moiety may consist of a polypeptide.

The protein 1 having a linker moiety 4 bound to the ligand moiety 2 and being capable of being coupled to a solid phase 8 allows for immobilisation of the protein 1 to a solid phase 8, as illustrated in FIG. 2. Such a solid phase 8 with bound protein 1 may be used as solid phase 8 material or affinity capture medium in affinity chromatographic applications. Thus, FIG. 2 may illustrate also an affinity capture medium according to the second aspect, or variants thereof. With further reference to FIG. 2, the linker moiety 4 provides a distance between the ligand moiety 2 and the solid phase 8, thus reducing risks of deformation, such as denaturation, of the ligand moiety 2, and further reducing risks associated with an active site of the ligand moiety 2 being sterically hindered or otherwise obscured from interaction or binding with a target molecule, such as a POI or a target analyte comprising a POI. The solid phase 8 is schematically illustrated as a surface, and it will be appreciated that the solid phase may be provided in any suitable form, such as a bead, a chip, a fibrous or nanofibrous structure, or a monolithic structure. Further, the solid phase 8 may be provided with magnetic properties, thereby facilitating, for example, control or manipulation of the solid phase 8. For example, the solid phase 8, may be in form of magnetic beads. Any suitable solid phase 8 that may be used as medium for chromatographic or separation purposes and being suitable for binding of the protein 1, as described herein, may be used. It will be appreciated that if the solid phase takes a form that has a convex surface or structure, for example, a bead or sphere, the linker moiety 4 allows higher number of ligand moieties 2 to be linked to the solid phase as compared to if the ligand moieties would be bound directly to the solid phase 8, or with a considerably shorter linker moiety 4. Porous medium also may allow higher number of ligand moieties 2 to be linked to the solid phase.

The linker moiety may comprises or consist of an amino acid sequence having 5 to 100 amino acid residues.

The linker moiety may comprise or consist of an amino acid sequence having 5 to 75, such as 5 to 50, 5 to 30, 9 to 30, or 12 to 20 amino acid residues.

The ligand moiety may comprise or consist of a peptide or protein. Thus, the ligand moiety may comprise or consist of an amino acid sequence.

The linker moiety may have an isoelectric point above 6.0. Thereby, improved binding to the solid phase may be realised.

The linker moiety may have at least three lysine residues. Thereby, improved binding to the solid phase may be achieved.

The linker moiety may have an isoelectric point above 8.0. In particular, if the linker moiety has three lysine residues, the linker moiety may have an isoelectric point above 8, thereby improved immobilisation of the protein to the solid phase may be achieved.

The ligand moiety may be a split N-intein or a split C-intein, thus capable of affinity interaction with a corresponding split C-intein or split N-intein, respectively.

The target molecule, e.g. a POI, may be fused with, for example expressed with, a split intein capable of interaction with the split intein of the ligand moiety.

The split N-intein and the split C-intein may be from a naturally split intein or an engineered split intein.

The ligand moiety may be a split N-intein and the linker moiety may be a C-terminally arranged linker moiety, wherein the split N-intein comprises the following sequence:

(SEQ ID NO: 1)
ALSYDTEILTVEYGFLPIGXIVEEXIEXTVYSVDXXGFVYTQPIAQWHN
RGEQEVFEYXLEDGSIIRATXDHXFMTTDGXMLPIDEIFEXGLDLXQV,

wherein,
X in positions 20, 35, 70, 73, and 95 are each independently selected from R or A;
X in position 28 is C, A or S;
X in position 36 is N, H or Q;
X in position 25 is N or R;
X is position 59 is D or C;
X in position 80 is E or Q; and
X in position 90 is Q, or R.

It is beneficial that such a split N-intein is free from lysine residues. When combined with the linker moiety having at least two lysine residues, specific binding to the solid phase via the linker moiety may be realised, in particular when at least one residue other than lysine is terminally arranged in relation to at least two of the lysine residues. Thereby, the ligand may be immobilised to the solid phase while reducing risks of deformation of the ligand moiety, allowing for improved affinity interaction and separation. The particular sequence has in addition been selected for having improved alkali stability which provides tolerance to increased pH values during for example chromatographic procedures, such as regeneration of the solid phase using high alkaline solution. Thereby, re-use of the solid phase, or re-use of a column used for affinity interaction may be realised.

Of SEQ ID NO: 1,
X in positions 20, 35, 70, 73, and 95 may be R;
X in position 28 may be A;
X in position 36 may be H;
X in position 25 may be N;
X in position 59 may be D;
X in position 80 may be E; and
X in position 90 may be Q.

Thus, the split N-intein may comprises or consist of the following sequence:

(SEQ ID NO: 2)
ALSYDTEILTVEYGFLPIGRIVEENIEATVYSVDRHGFVYTQPIAQWHN
RGEQEVFEYDLEDGSIIRATRDHRFMTTDGEMLPIDEIFEQGLDLRQV.

Further provided is an N-intein protein variant of the native N-intein domain of Nostoc punctiforme (Npu) wherein the native N-intein domain has the sequence defined by SEQ ID NO: 21.

With the protein for affinity capture according to the variant of the first aspect, wherein the ligand moiety is a split N-intein and the linker moiety is a C-terminally arranged linker moiety, the split N-intein may comprise or consist of a sequence having at least 70% or 80% identity with SEQ ID NO: 21, such as at least 90% or 95% identity. For such a split N-intein, the position 36 of SEQ ID NO: 21 may suitably be substituted with Histidine, H, and any present Lysine substituted with an amino acid other than Lysine, thereby allowing provision of a ligand moiety being free from lysine residues. Lysine may suitably be replaced with Arginine, which retains the charge and charge properties of the split N-intein.

The linker moiety may comprise an amino acid sequence selected from the list consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12

This particular plurality of sequences (SEQ ID NO: 3-12) has been chosen from a larger plurality of sequences for providing comparably improved immobilisation to a solid phase. It shall be noted that each of those sequences comprises at least two lysine residues, wherein at least one residue other than lysine is terminally arranged in relation to at least two of the lysine residues, which provides for improved immobilisation to the solid phase. Put differently, the at least two of the lysine residues must have at least one amino acid residue other than lysine, i.e. a non-lysine amino acid residue, in the terminal direction. In particular, the linker moiety may be described by SEQ ID NO: 3.

An example of the linker moiety being directly bound to the ligand moiety may be illustrated, for example, using SEQ ID NO: 3 fused with an N-intein according to SEQ ID NO: 2 together forming a protein for affinity capture described by SEQ ID NO: 20.

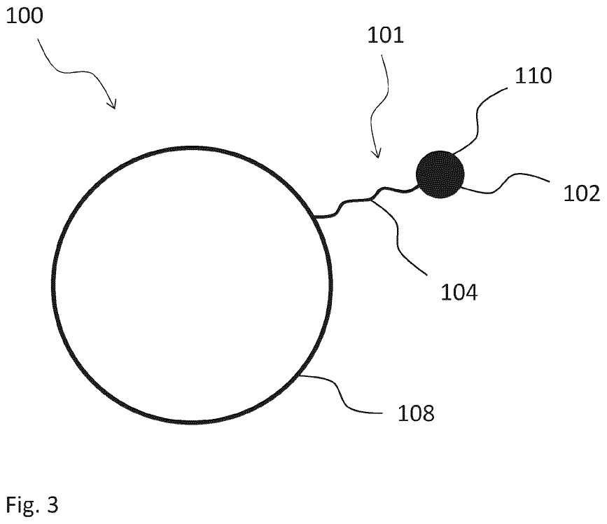

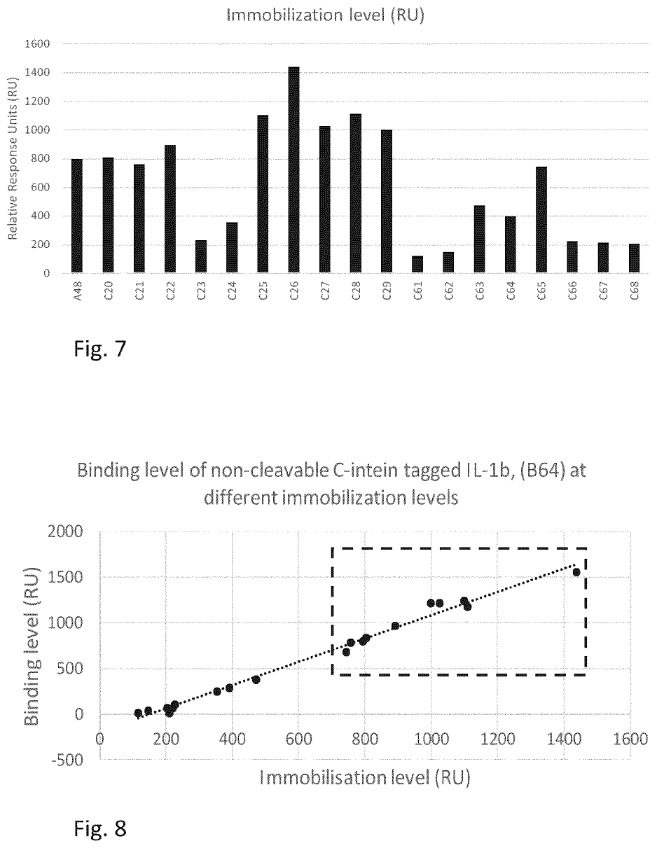

With reference to FIG. 3, an affinity capture medium 100 as described by the second aspect is schematically illustrated. The affinity capture medium 100 comprises the protein 101 for affinity capture according to the first aspect, and a solid phase 108. The ligand moiety 102 of the protein is covalently attached to the solid phase 108 via the linker moiety 104. FIG. 3 illustrates an example wherein the solid phase 108 is illustrated as a sphere, although it shall be appreciated that other suitable shapes are possible. The ligand moiety 102 is illustrated as having an interaction site 110, which is capable of affinity interaction with a target molecule. It shall be understood that although FIG. 7 is schematically illustrated with only one protein 101 for affinity capture bound to the solid phase 108, it is achievable, and may be beneficial to have several proteins 101 bound to a solid phase 108 structure.

The linker moiety 104 of the protein 101 may be covalently attached to the solid phase via at least one lysine residue of the amino acid sequence of the linker moiety. The linker moiety 104 may be covalently attached to a solid phase 108 by chemical reaction involving contacting between lysine residues and the solid phase 108. Examples of chemistries or reagents suitable for reaction with lysine residues include, for example, cyanogen bromide, epoxides, or N-hydroxysuccinimide esters.

The solid phase may be in form of a bead, a membrane, a particle, a chip, a fibrous or nanofibrous structure, or a monolithic structure, or combinations thereof.

In particular, the solid phase may be in form of a bead.

The solid phase may comprise or be composed of a natural or synthetic polymeric material, preferably a polysaccharide such as cellulose or agarose.

The solid phase may comprise or be composed of one or more compounds selected from the group consisting of agarose, polystyrene, methacrylate and cellulose, or combinations thereof.

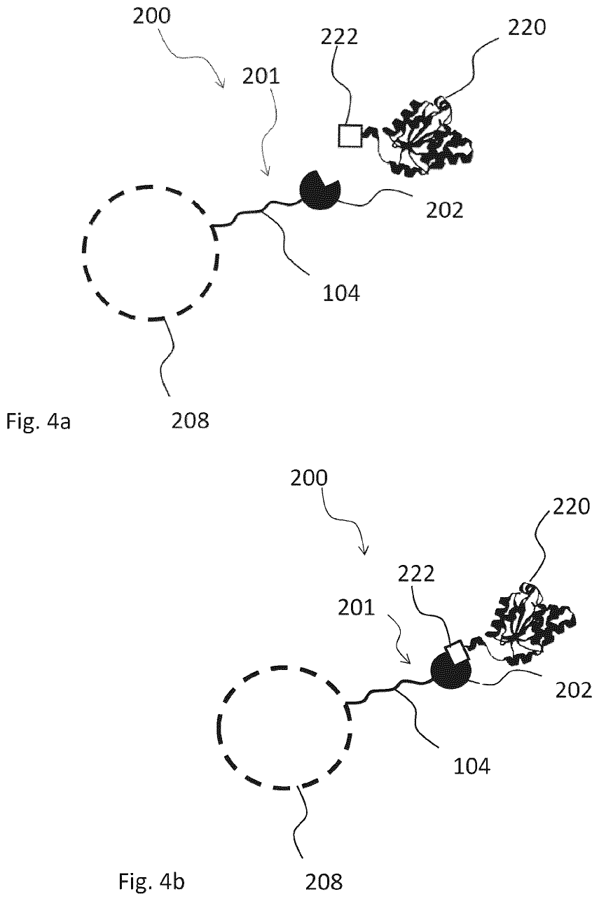

With reference to FIGS. 4a and 4b, a split intein system 200 for affinity capture of a protein of interest 220 (POI) according to a variant of the third aspect will now be discussed. The split intein system 200 comprises a protein 201 for affinity capture according to the first aspect, wherein the ligand moiety 202 is a split N-intein. The split intein system 200 further comprises a target molecule which, for example, may be a POI fused with a split C-intein 222. The split C-intein 222 may, for example, be expressed in fusion with the POI 220. The split N-intein and the split C-intein 222 are sequences of a split intein. The protein 201 is capable of being coupled, such as via covalent attachment or binding, to a solid phase 208, which is illustrated using dotted lines of the solid phase 208. For example, the covalent attachment or binding may be accomplished using chemistries discussed above. FIG. 4a illustrates the split intein system 200 wherein the target molecule, which is a POI fused with the split C-intein 222, has not yet interacted with the protein 201. FIG. 4b illustrates the split intein system 200 wherein the target molecule which is a POI fused with the split C-intein 222, has contacted and interacted with the protein 201, via the split C-intein 222.

Using a split intein as described above provides several benefits. The affinity recognition and interaction between the split N-intein and the split C-intein of a split intein provides for high selectivity in the separation. Further, inherent properties of inteins allows for spontaneous cleavage, or self-cleavage, whereby the POI is cleaved off the split intein with which it is fused, thereby allowing for efficient elution of the POI in a separation or chromatographic method. The split C-intein may be dissociated from the split N-intein under regeneration of the protein 201 or the affinity capture medium comprising the protein 201.

The protein for affinity capture may be comprised in an affinity capture medium according to the second aspect.

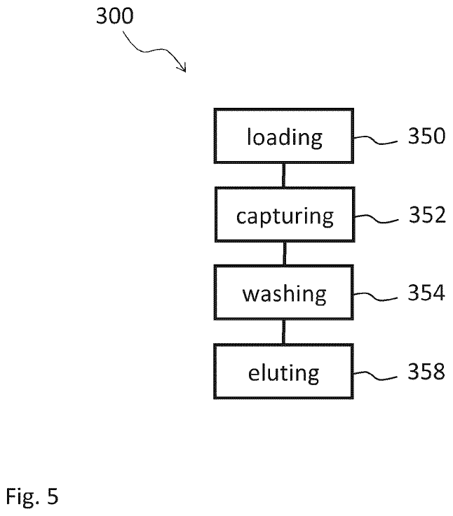

With reference to FIG. 5, a method 300 for purification of a protein of interest (POI) according to a variant of the further aspect will now be discussed. The method 300 comprises: loading 350 a sample liquid comprising the POI on an affinity capture medium comprising a protein for affinity capture, having affinity for the POI or for a target molecule comprising the POI, covalently attached to a solid phase via a linker moiety, wherein the protein for affinity capture is the protein according to the first aspect; capturing 352 the POI or the target molecule comprising the POI by binding to the protein of the affinity capture medium; washing 354 the affinity capture medium with washing liquid to remove compounds other than the POI or the target molecule comprising the POI; and eluting 358 the POI or the target molecule comprising the POI from the affinity capture medium.

The POI or the target molecule comprising the POI may be released from the protein by use of a split intein system and an inherent capability of inteins to cleave off a fused protein, e.g. the POI.

The affinity capture medium may be the affinity capture medium according to the second aspect.

The target molecule comprising the POI may, for example, be a target molecule which is a POI fused with a peptide, or the target molecule comprising the POI may be the POI, for which case a ligand moiety suitable for affinity interaction with the POI may be selected for the protein for affinity capture.

The affinity capture medium according to the fourth aspect may be the affinity capture medium according to the second aspect.

The protein for affinity capture may a protein according to the first aspect, wherein the ligand is a split N-intein, and the target molecule is a POI fused with a split C-intein.

It will be appreciated that although the method 300 and variants of the fourth aspects describes purification of a POI, alternative variants may relate to purification of other molecules of interest that are suitable to purify with such a method.

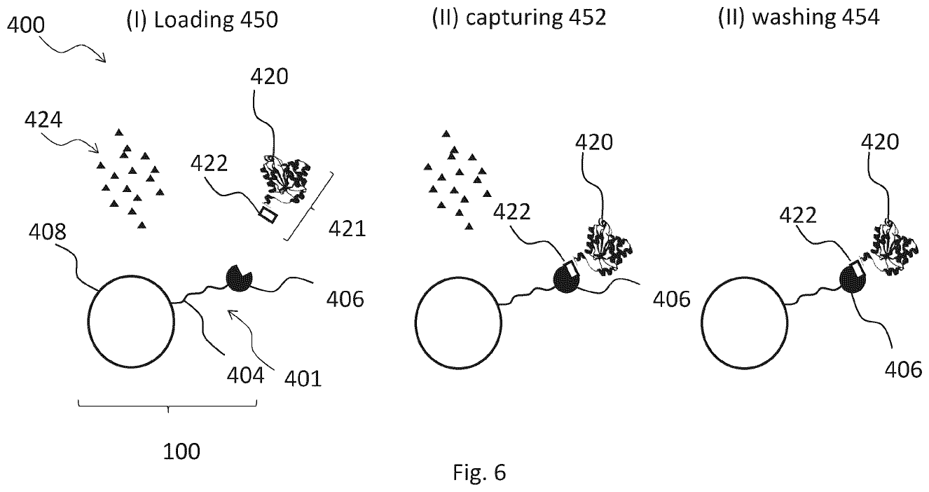

With reference to FIG. 6, a method 400 for purification of a POI according to a variant of the method discussed with reference to FIG. 5 will now be discussed in further detail. The method 400 comprises use of a protein 401 for affinity capture according to variants of the first aspect, in other words a protein 401 for affinity capture comprising a ligand moiety 402 capable of affinity interaction with a target molecule 421, for this example a POI 420 fused with an target molecule moiety 422, although other target molecules alternatively may be compatible and usable for the method 400. For the example of the target molecule 421 comprising the POI 420, the target molecule 421 may comprise a target molecule moiety 422 being capable of affinity interaction with the ligand moiety 402. The protein 401 for affinity capture further comprises a linker moiety 404 bound to the ligand moiety 402 and capable of being coupled to a solid phase 408. The ligand moiety 402 of the example is free from lysine residues, and the linker moiety 404 is terminally positioned in the protein 401. Further, the linker moiety comprises an amino acid sequence having 5 to 100 amino acid residues, of which amino acid residues at least two are lysine residues. Further, at least one residue other than lysine is terminally arranged in relation to at least two of the lysine residues.

The method 400 comprises (I) loading 450 a sample liquid, for the present example a water solution, comprising the target molecule 421 comprising the POI 420 on a affinity capture medium 100 comprising the protein 401 for affinity capture having affinity for the target molecule comprising the POI covalently attached to the solid phase 208 via the linker moiety 404. The loading 450 may be, for example, injection of the sample liquid into a chromatographic column or on a chromatographic membrane (a column or a membrane is not illustrated in FIG. 6). The sample liquid may be, for example, a cell culture or obtained from a cell culture, such as a cell culture supernatant or lysate. Such sample liquids typically may be considered to comprise considerable amounts of contaminants or molecules other than the target molecule. The sample liquid may have been subjected to sample clean-up or purification before sample loading. For the purpose of sample loading and binding, a suitable binding buffer may be present, for example, the sample liquid may be sample mixed with sample buffer. The sample buffer or sample liquid may comprise buffering agents and/or salt. The sample liquid or binding buffer may have a pH within a range of 6-9. The salt concentration may be within a range of 0-1 M, such as 1 mM-1 M. The binding buffer or sample liquid may comprise NaCl and or phosphate salt. Phosphate salt may be used for adjusting or setting the pH value. NaCl may suitably be used for adjusting salt concentration. For example, the binding buffer may comprise or consist of phosphate buffered saline (PBS) at pH 7.4. The method 400 further comprises (II) capturing 452 the target molecule 421 comprising the POI 420 by binding to the protein 401 of the affinity capture medium 100. Binding of the target molecule 421 comprising the POI 420 to the protein 401, may be achieved by allowing contacting between the target molecule 421 comprising the POI 420 and the protein 401, for example achieved or contributed to by diffusion of the target molecule 421 comprising the POI within the sample liquid. As an example, if the target molecule 421 is a POI fused with a split C-intein, such as being expressed in fusion with 5 the split C-intein, and the ligand moiety comprises a corresponding split N-intein, the capturing 452 may involve affinity interaction between the split C-intein and the split N-intein. Thereafter, (Ill) washing 354 the affinity capture medium with washing liquid to remove compounds other than the target molecule comprising the POI 420 from the sample liquid is conducted. Compounds 424 other than the POI 420, which compounds 424 are schematically represented by triangles in FIG. 6, may be any compounds present in the sample liquid other than the target molecule, for example the POI, such as, for example, other proteins, DNA, RNA, salts, or reagents. As such, the other compounds 424 than the target molecule may be viewed as contaminants or impurities. Resulting from the specificity of the protein 401 towards the target molecule 421 comprising the POI 420, compounds 424 will not bind to the protein 401 via affinity interaction during or after loading 450. However, contaminants or compounds may interact with the solid phase 408 or the affinity capture medium 100 using other mechanisms, such as one or more mechanisms based on, for example, hydrogen bonds, ion interaction, hydrophobic effects, or hydrophilic interaction. The washing 354 may remove at least a major portion of the other compounds 424 from the affinity capture medium 100. The washing 354 may be conducted using a suitable buffer, which may be aqueous, and which may comprise salt and/or washing agents. By selecting a ligand moiety 402 capable of handling high pH solution, or alkali solution, washing may be conducted using high pH, which may provide efficient removal of other compounds than the POI. If split inteins are used, Zn²⁺-ions may be present in the wash buffer to prevent elution of the target molecule comprising the POI during washing.

After washing 454, follows eluting 456 the POI or the target molecule comprising the POI from the affinity capture medium. The eluting 456 may be, at least in part, realised or preceded by waiting over a holding time, for example, if a self cleaving split-intein is used as ligand moiety and a corresponding split-intein is expressed in fusion with the POI 420. The holding time may be, for example, between 1 and 30 hours, such as between 1 to 25 hours; or 2 to 6 hours, or 15 to 25 hours, such as around 4 hours or around 20 hours. Then follows eluting the POI or the target molecule comprising the POI from the affinity capture medium, thereby obtaining the POI purified from compounds from the sample liquid. For example, elution may be promoted or conducted by changes in properties of present buffer or liquid. For example, the pH or ionic or salt concentration, or hydrophobicity or hydrophilicity, or combinations of different such properties may be changed to promote elution.

For example, elution may be conducted using a solution comprising or consisting of imidazole in PBS, for example, 250 mM imidazole in PBS adjusted to pH 7.4.

Following elution, regeneration of the solid phase, or a column comprising the solid phase, may be conducted. Regeneration may comprise washing with regeneration liquid, which may be alkaline solution. Regeneration of the solid phase, for example comprised in a column, may be used for preparing the solid phase or column for additional purification of a protein of interest (POI).

Thus, the method 400 for purification of a protein of interest (POI) may further comprise regeneration of the solid phase comprising washing with regeneration liquid.

Regeneration may, for example, be conducted using a regeneration liquid comprising or consisting of an aqueous solution of sodium hydroxide and urea, for example 100 mM sodium hydroxide and 4 M urea.

The ligand moiety of this variant of the fourth aspect may be a split N-intein and the linker moiety may be a C-terminal linker moiety, wherein the split N-intein comprises the sequence defined by SEQ ID NO: 2.

The C-terminal linker moiety may comprise an amino acid sequence selected from the list consisting of SEQ ID NO: 3-12.

It will, thus, be appreciated that the methods 300, 400 may use a split intein system for affinity capture of a POI, comprising a protein for affinity capture according to the first aspects and variants thereof, wherein the ligand is a split N-intein; and a split C-intein which is expressed in fusion with the POI.

The ligand moiety of these variants of the fourth aspect illustrated with reference to FIGS. 5 and 6 may be covalently attached to the solid phase 408 via lysine residues of the linker moieties, thus forming an affinity capture medium.

The sample liquid may be any suitable liquid comprising the POI. For example, the sample liquid may be or be obtained from a cell culturing medium in which the POI has been expressed. The sample may be an aqueous sample liquid, or a water solution.

### EXPERIMENTS AND EXAMPLES

Experiments were conducted to evaluate immobilisation of proteins for affinity capture on a solid phase. An aim was to achieve high surface concentration of the proteins on the solid phase, as a high surface concentration provides desirable separation or chromatographic properties. A further aim was to achieve efficient binding of target molecules using the solid phase with immobilised proteins.

The protein for affinity capture used for the experiments comprised a ligand moiety capable of affinity interaction with a target molecule, and a linker moiety bound to the ligand moiety. Immobilisation on a solid phase was achieved by binding of the linker moiety on the solid phase via covalent linking of lysine residues to the solid phase.

The experimental set ups were based on a realisation that a ligand moiety being free from lysine residues, while having a linker moiety having an amino acid sequence comprising lysine may result in efficient binding to the solid phase via the linker moiety.

### Ligand Moiety

For the experiments, ligand moieties having the following sequence were used:

(SEQ ID NO: 2)
ALSYDTEILTVEYGFLPIGRIVEENIEATVYSVDRHGFVYTQPIAQWHN
RGEQEVFEYDLEDGSIIRATRDHRFMTTDGEMLPIDEIFEQGLDLRQV.

The ligand moieties used for the experiments may further be described as being N-inteins.

### Linker Moiety

For the experiments, the ligand moieties, being N-inteins, were designed and, at the C-terminal, provided with different linker moieties in form of amino acid sequences of varying lengths and including variations in number of lysine residues and position of the lysine residues.

As such, the ligand moieties provided with the linker moieties may be considered as being proteins comprising a ligand moiety and a linker moiety bound to the ligand moiety wherein the ligand moiety comprises a ligand protein or a ligand peptide free from lysine residues, and wherein the linker moiety is terminally positioned in the protein and comprises an amino acid sequence.

Amino acid sequences of the linker moieties used for the experiments are provided in table 1.

TABLE 1

| Identi- | | | No. of | |
|---|---|---|---|---|
| fying | | | amino | |
| name | Amino acid sequence* | pI** | acids | SEQ ID |
| A48 | DADSGGSGKDDAKKKDDTKK | 6.18 | 20 | (SEQ ID NO: 3) |
| C20 | GGSGKDDAKKKDDTKK | 9.31 | 16 | (SEQ ID NO: 4) |
| C21 | DADSKDDAKKKDDTKK | 6.18 | 16 | (SEQ ID NO: 5) |
| C22 | KDDAKKKDDTKK | 9.31 | 12 | (SEQ ID NO: 6) |
| C23 | DADSGGSGK | 4.21 | 9 | (SEQ ID NO: 13) |
| C24 | DADSGGSGKDDAKKK | 6.10 | 15 | (SEQ ID NO: 14) |
| C25 | ARTKQTARKSTGGK | 12.02 | 14 | (SEQ ID NO: 7) |
| C26 | ARTKQTARKSTGGKAPRK | 12.31 | 18 | (SEQ ID NO: 8) |
| C27 | SGRGKGGKGLGKGGAK | 11.33 | 18 | (SEQ ID NO: 9) |
| C28 | AKSAPAPKKGSKK | 10.6 | 13 | (SEQ ID NO: 10) |
| C29 | PKKTESSKSKSK | 10.18 | 12 | (SEQ ID NO: 11) |
| C61 | DADSGGSGDK | 3.93 | 10 | (SEQ ID NO: 15) |
| C62 | DADSGGSGKDDTKK | 4.58 | 14 | (SEQ ID NO: 16) |
| C63 | ARTRQTARKSTGGK | 12.31 | 14 | (SEQ ID NO: 17) |
| C64 | ARTKQTARRSTGGK | 12.31 | 14 | (SEQ ID NO: 18) |
| C65 | ARTKQTARKSTGGR | 12.31 | 14 | (SEQ ID NO: 12) |
| C66 | K | 8.75 | 1 | — |
| C67 | KDK | 8.59 | 3 | — |
| C68 | DYKDDDDK | 3.97 | 8 | (SEQ ID NO: 19) |

*The displayed amino acid sequences are for the linker moiety, which is coupled to a ligand moiety, at the C-terminal of the ligand moiety, defined according to SEQ ID NO: 2.
**pI values are estimated using http://www.expasy.org/tools/pi_tool.html..

### Evaluation of Immobilisation of Ligand Moieties on a Solid Phase

Different proteins for affinity capture were evaluated. All evaluated proteins each comprised the ligand moiety described by SEQ ID NO: 2, as discussed above, and one of the linker moieties of table 1. The evaluation used a Biacore T-200 instrument, (Cytiva, Sweden), using surface plasmon resonance, SPR, for detection and analyses. The proteins comprising the ligand moiety and the respective linker moiety were purified prior to SPR analyses to >95% purity after expression in E. coli BL21(DE3) by using a combination of hydrophobic interaction chromatography and ion exchange chromatography, (HiTrap Phenyl Sepharose HP and Capto Q respectively, Cytiva. Purity was determined using CBB-stained, Coomassie brilliant blue, SDS-PAGE polyacrylamide gels, (SERVA Electrophoresis GmbH), and size-exclusion chromatography analysis on a Superdex 75 Increase (Cytiva). Identification of each protein comprising the ligand moiety and respective linker moiety was confirmed by comparing theoretical molecular weight from the amino acid sequence and LC-MS full-length analysis data, (BioAccord System, Waters). C-intein tagged proteins that were used for test separations were purified by Strep-Tactin XT Sepharose then analyzed with the same methods as the proteins comprising the ligand moiety and the respective linker moiety. Biacore methods were performed at 25° C. with HBS-EP+, pH 7.4, (Cytiva AB) as running buffer.

All reagents and buffers were from Cytiva and the user instructions and standard settings were followed. Immobilization of the proteins comprising the ligand moiety and the respective linker moiety were conducted using series-S sensor chip CM5 as solid phase by activating the surface of the chip using EDC and NHS from the Amine coupling kit, then contacting the activated surface by injecting purified ligand moieties with coupled linker moieties, diluted to 20 μg/ml with 10 mM Na-acetate pH 4.0. sample injection was done for 420 seconds at 10 μl/min. Finally, remaining unreacted NHS groups were blocked by injecting 1M Ethanolamine. The response, RU or resonance units, is directly proportional to the concentration of proteins on the surface. The immobilization levels were determined by subtracting the absolute response prior to immobilization from the absolute response after immobilization, equal to the relative response.

The relative responses from immobilization of different linker-ligand pairs of the proteins are shown in FIG. 7, and also displayed in table 2. It can be concluded from the data of the experiments as illustrated in FIG. 7, together with the sequences of table 1, that if the linker moiety has at least two lysine residues, wherein at least one residue other than lysine is terminally arranged in relation to at least two of the lysine residues higher immobilisation levels were achieved.

It can further be concluded that the linker moieties having an isoelectric point (pl) above about 6, in particular a pl above above 8, may provide beneficial immobilisation levels.

### Evaluation of Binding Capacity for the Immobilised Ligand-Linker Pairs

The relative binding capacity of each ligand-linker pair (of table 1) was tested after immobilization of the ligand moieties to the sensor chips. The capacity was evaluated using a mutant, non-cleavable split C-intein tagged test-protein B64, (Interleukin 1-b, IL-1b). Self-cleaving functionality was inactivated by a point mutation of the catalytic C-terminal Asn in the C-intein sequence to Ala, (N36A). Purified test-protein B64 was diluted to 20 μg/ml with HBS-EP+, pH 7.4 buffer, followed by sample injection to the sensor chip at 30 μl/min for 1 minute. The relative response was measured 10 seconds after sample injection. Binding levels are shown as relative response in table 2, in which table 2 also immobilisation levels from table 1 are displayed.

TABLE 2
Binding levels, immobilisation levels, and number of lysine
residues of different linker moieties of the proteins.

| | | | | Presence of at |
|---|---|---|---|---|
| | | | | least 2 lysine |
| | | | | residues that |
| | | | Total | are not |
| | | Binding | number | terminally |
| Identifying | Immobilization | level | of lysine | arranged |
| name | level (RU) | B64 (RU) | residues¹ | [Yes/No]² |
| A48* | 794 | 801 | 6 | Yes |
| C20* | 804 | 841 | 6 | Yes |
| C21* | 758 | 787 | 6 | Yes |
| C22* | 890 | 968 | 6 | Yes |
| C23 | 227 | 102 | 1 | No |
| C24 | 354 | 253 | 4 | No |
| C25* | 1100 | 1241 | 3 | Yes |
| C26* | 1438 | 1556 | 4 | Yes |
| C27* | 1023 | 1213 | 4 | Yes |
| C28* | 1108 | 1173 | 5 | Yes |
| C29* | 999 | 1212 | 5 | Yes |
| C61 | 117 | 11 | 1 | No |
| C62 | 146 | 39 | 3 | No |
| C63 | 471 | 376 | 2 | No |
| C64 | 391 | 290 | 2 | No |
| C65* | 742 | 674 | 2 | Yes |
| C66 | 220 | 67 | 1 | No |
| C67 | 210 | 9 | 2 | No |
| C68 | 202 | 60 | 2 | No |

¹Total number of lysine residues in linker moiety.
²“Yes” indicates that the linker moiety has at least two lysine residues, wherein at least one residue other than lysine is terminally arranged in relation to at least two of the lysine residues. “No” indicate that the linker moiety has less than two lysine residues having at least one residue other than lysine terminally arranged in relation to the lysine residues.

In table 2, the linker moieties having at least two lysine residues, wherein at least one residue other than lysine is terminally arranged in relation to at least two of the lysine residues are marked using asterisks with the identifying name. Notably, those the linker moieties were associated with the highest immobilisation levels.

FIG. 8 illustrates the binding level versus the immobilisation levels, using data from table 2.

It can be concluded from table 2 and from FIG. 8 that there is a clear and strong correlation between immobilization levels and binding levels. Further, it may be concluded that the linker moieties resulting in the highest immobilisation levels also have the highest, and thus most desirable, binding levels. In FIG. 8, the linker moieties having at least two lysine residues, wherein at least one residue other than lysine is terminally arranged in relation to at least two of the lysine residues, i.e. the linker moieties having the highest immobilisation levels, are marked using a dotted lined square.

### Experiment—Column Application

An N-intein protein consisting of a ligand moiety according to SEQ ID NO: 2 and a linker moiety according to SEQ ID NO: 7 (identifying name C25), was purified to >95% purity after expression in E. coli BL21(DE3) by using a combination of hydrophobic interaction chromatography and ion exchange chromatography, (HiTrap Phenyl Sepharose HP and Capto Q respectively, Cytiva). Purity was determined by Coomassie brilliant blue stained SDS-PAGE gels, (SERVA Electrophoresis GmbH), and size-exclusion chromatography analysis on a Superdex 75 Increase column (Cytiva). Identification of the N-intein protein was confirmed by comparing the experimental molecular weight obtained from mass spectrometry data, (BioAccord System, Waters) with the theoretical molecular weight calculated from the amino acid sequence. The N-intein protein was further concentrated by ultrafiltration to a concentration of 41 mg per ml in a buffer containing 50 mM sodium-phosphate and 0.5 M sodium chloride adjusted to pH 7.0. The pH was adjusted to pH 10-11 prior to coupling to epoxy-activated solid phase in form of agarose beads. 8 grams of epoxy-activated agarose beads were added into a small 50 ml reactor flask. 3.7 ml ligand in solution was added. The pH of the resulting reaction mixture was adjusted to pH 11 with 2 M sodium hydroxide. The reaction mixture was mixed for 3 hours. The resulting agarose bead slurry was transferred to a glass filter and washed with 10 ml of distilled water 5 times.

After washing, the gel resin was transferred into a small 50 ml reactor flask and 8 ml 0.1 M Tris buffer pH 10 and 0.6 ml thioglycerol was added. The pH was adjusted to pH 8.5. The reaction mixture was stirred for 2 hours. The resulting agarose bead slurry was transferred to a glass filter and washed with 0.1 M Tris buffer and then with 0.5 M Acetic acid. This base/acid wash was repeated another 2 times. Then the gel resin was washed with distilled water. The washed and drained gel resin was finally kept in 20% ethanol before packing 1 ml columns.

The receptor binding domain, RBD, from SARS-CoV-2 spike protein was used as a test-protein. The test-protein, B58 with a C-terminal polyhistidine tag and a N-terminal signal-sequence and a C-intein tag was expressed in Expi293 cells and secreted into the cell culture medium. 6 days post transfection the cell culture medium was harvested followed by tangential flow filtration to remove cells and particles. The final sample was stored at −80° C. until purification.

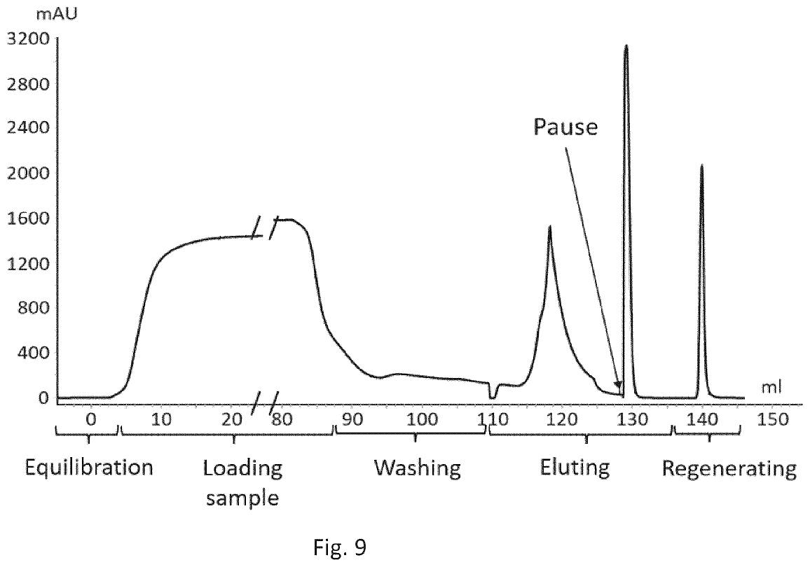

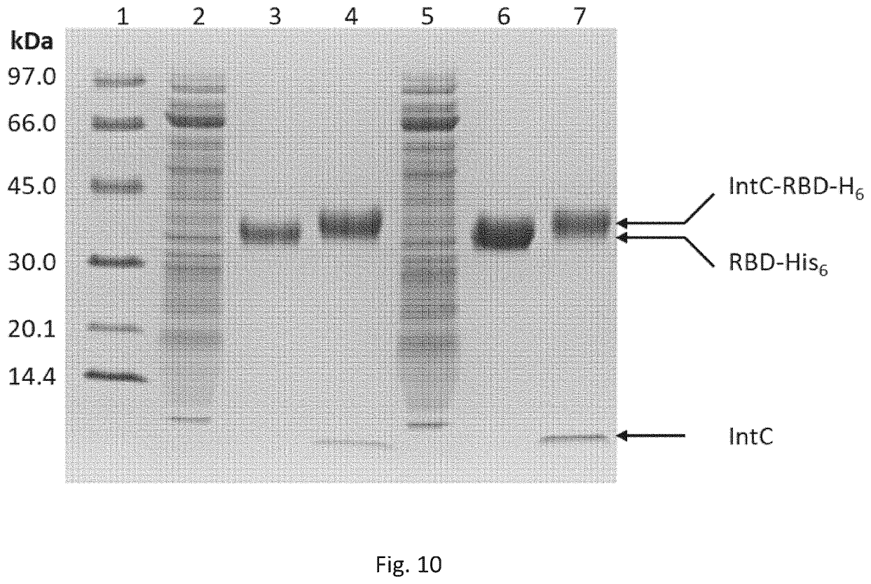

A UV-chromatogram covering the steps from equilibration to regeneration of the column is presented in FIG. 9. The sample containing the test-protein B58 was thawed and applied to a tandem column setup, a 5 ml HisTrap Excel, (Cytiva) was connected in series to the 1 ml column containing immobilized N-intein protein. Equilibration was conducted using an equilibration buffer in form of phosphate buffered saline, PBS at pH 7.4 and the flowrate was 1 ml/min during the loading of 80 ml sample and then 2 ml/min during washing and eluting. After elution with 12 column volumes of an elution buffer containing 250 mM imidazole in PBS adjusted to pH 7.4, the flowrate was stopped to allow for cleavage incubation during a 20 hours pause, indicated in FIG. 9 with “Pause”. Cleaved protein was eluted as a sharp peak in the UV-chromatogram after incubation as indicated by the arrow in FIG. 9, and was collected for further analysis. Finally, the column was regenerated with 100 mM sodium hydroxide containing 4 M urea, which resulted in a sharp peak at the end of the chromatogram. Results from the purification are shown in FIG. 9, (chromatogram) and FIG. 10, (SDS-PAGE analysis). The amount of eluted RBD estimated from UV-absorbance at 280 nm was 10.6 mg and 67% of the fusion protein was recovered whereas 33% was found in the regeneration fraction.

The experiment was repeated but the cleavage incubation time after sample application, washing and elution was changed to 4 hours instead of 20 hours. The cleaved and eluted amount of RBD was lower as expected compared with the longer incubation time. The amount of eluted RBD estimated from UV-absorbance at 280 nm was 7.7 mg and 46% of the fusion protein was recovered whereas 54% was found in the regeneration fraction.

The purity of the eluted RBD from both purifications were close to 100% according to SDS-PAGE. A photograph of the SDS-PAGE gel is depicted in FIG. 10. Data obtained from the SDS-PAGE gel is presented in table 3.

TABLE 3
Data obtained from SDS-PAGE gel of FIG. 10.

| Lane | Sample | Dilution x |
|---|---|---|
| 1 | LMW, size marker | 1 |
| 2 | Flow-through | 1 |
| 3 | Elution after 4 h pause | 30 |
| 4 | Regeneration after elution | 30 |
| 5 | Flow-through | 1 |
| 6 | Elution after 20 h pause | 15 |
| 7 | Reg. after elution | 15 |

1. A protein for affinity capture, the protein comprising
a ligand moiety capable of affinity interaction with a target molecule, and
a linker moiety bound to the ligand moiety and capable of being coupled to a solid phase,
wherein the ligand moiety is free from lysine residues, and
wherein the linker moiety is terminally positioned in the protein and has at least two lysine residues, wherein, in the linker moiety, at least one residue other than lysine is terminally arranged in relation to at least two of the lysine residues.

2. The protein for affinity capture according to claim 1, wherein the linker moiety comprises an amino acid sequence having 5 to 100, preferably 5 to 30, amino acid residues.

3. The protein for affinity capture according to claim 1, wherein the linker moiety has an isoelectric point above 6.0.

4. The protein for affinity capture according to claim 1, wherein the linker moiety has at least three lysine residues.

5. The protein for affinity capture according to claim 1, wherein the ligand moiety is a split N-intein or a split C-intein, thus capable of affinity interaction with a corresponding split C-intein or split N-intein, respectively.

6. The protein for affinity capture according to claim 1, wherein the ligand moiety is a split N-intein and the linker moiety is a C-terminally arranged linker moiety, wherein the split N-intein comprises the following sequence:
(SEQ ID NO: 1)
ALSYDTEILTVEYGFLPIGXIVEEXIEXTVYSVDXXGFVYTQPIAQWHN
RGEQEVFEYXLEDGSIIRATXDHXFMTTDGXMLPIDEIFEXGLDLXQV,
wherein,
X in positions 20, 35, 70, 73, and 95 are each independently selected from R or A;
X in position 28 is C, A or S;
X in position 36 is N, H or Q;
X in position 25 is N or R;
X is position 59 is D or C;
X in position 80 is E or Q; and
X in position 90 is Q, or R.

7. The protein for affinity capture according to claim 5, wherein
X in positions 20, 35, 70, 73, and 95 is R;
X in position 28 is A;
X in position 36 is H;
X in position 25 is N;
X in position 59 is D;
X in position 80 is E; and
X in position 90 is Q.

8. The protein for affinity capture according to claim 1, wherein linker moiety comprises an amino acid sequence selected from the list consisting of:
(SEQ ID NO: 3)
DADSGGSGKDDAKKKDDTKK,
(SEQ ID NO: 4)
GGSGKDDAKKKDDTKK,
(SEQ ID NO: 5)
DADSKDDAKKKDDTKK,
(SEQ ID NO: 6)
KDDAKKKDDTKK,
(SEQ ID NO: 7)
ARTKQTARKSTGGK,
(SEQ ID NO: 8)
ARTKQTARKSTGGKAPRK,
(SEQ ID NO: 9)
SGRGKGGKGLGKGGAK,
(SEQ ID NO: 10)
AKSAPAPKKGSKK,
(SEQ ID NO: 11)
PKKTESSKSKSK,
and
(SEQ ID NO: 12)
ARTKQTARKSTGGR.

9. An affinity capture medium comprising the protein for affinity capture according to claim 1, and a solid phase, wherein the ligand moiety of the protein is covalently attached to the solid phase via the linker moiety.

10. The affinity capture medium according to claim 9, wherein the linker moiety of the protein is covalently attached to the solid phase via at least one lysine residue of the amino acid sequence of the linker moiety.

11. The affinity capture medium according to claim 9, wherein the solid phase is in form of a bead, a membrane, a particle, a chip, a fibrous or nanofibrous structure, or a monolithic structure, or combinations thereof.

12. The affinity capture medium according to claim 9, wherein the solid phase comprises or is composed of a natural or synthetic polymeric material, preferably a polysaccharide such as cellulose or agarose.

13. A split intein system for affinity capture of a protein of interest (POI), comprising
a protein for affinity capture according to claim 1, wherein the ligand is a split N-intein, and
a target molecule which is a POI fused with a split C-intein.

14. The split intein system according to claim 11, wherein the protein for affinity capture is comprised in an affinity capture medium.

15. A method for purification of a protein of interest (POI) the method comprising:
loading a sample liquid comprising the POI on an affinity capture medium comprising a protein for affinity capture, having affinity for the POI or for a target molecule comprising
the POI, covalently attached to a solid phase via a linker moiety, wherein the protein for affinity capture is the protein according to claim 1,
capturing the POI or the target molecule comprising the POI by binding to the protein of the affinity capture medium,
washing the affinity capture medium with washing liquid to remove compounds other than the POI or the target molecule comprising the POI, and
eluting the POI or the target molecule comprising the POI from the affinity capture medium.

16. The method for purification of a protein of interest (POI), according to claim 12, wherein the affinity capture medium is the affinity capture medium.

17. The method for purification of a protein of interest (POI), according to claim 15, wherein
the protein for affinity capture is a protein, wherein
the ligand is a split N-intein, and
the target molecule is a POI fused with a split C-intein.
